(19) United States
(12) Plant Patent Application Publication
(10) Pub. No.: US 2005/0193454 P1
(43) Pub. Date: Sep. 1, 2005

(54) Variety of Hosta plant named 'BERSUN'

(50) HOSTA MARGINATA-ALBA; HOSTA

(76) Inventor: Leo Berbee, AD Lisse (NL)

Correspondence Address: FULWIDER PATTON LEE & UTECHT, LLP, 200 OCEANGATE, SUITE 1550, LONG BEACH, CA, 90802, US

(21) Appl. No.: 10/789,482

(22) Filed: Feb. 27, 2004

(51) Int. Cl. A01H 5/00

(52) U.S. Cl. PLT/353

(57) ABSTRACT

A new and distinct cultivar of Hosta plant named ‘BERSUN’ characterized by its relatively compact growth habit with a medium green foliage highlighted by a soft chartreuse variegation pattern and an abundance of pale lavender flowers.

### BACKGROUND OF THE NEW PLANT

The present invention relates to a new and distinct cultivar of Hosta, botanically known as Hosta fortunei marginata-alba and further known by the varietal name ‘BERSUN’. This new variety is also sold under the trade name SUNDANCER (Breeder's Ref. No. H 90-46). The new variety was discovered in a selective breeding program by the Inventor, Leo Berbee, in Lisse, The Netherlands in 1990. The new variety is a sport selection from a Hosta fortunei marginata-alba. The new cultivar was selected from the progeny of this sport selection in a controlled environment by the inventor in Lisse, The Netherlands. These divisions were selected and propagated through rhizome divisions selecting for differences in growth habit and distinctive leaf color pattern.

Continued observation of the vegetative divisions resulting from asexual reproduction of clump divisions taken at Lisse, The Netherlands have confirmed that the distinguishing features of this new cultivar came true, remain stable and are retained through successive propagations.

### SUMMARY OF THE INVENTION

Plants of the cultivar ‘BERSUN’ have not been observed under all possible environmental and cultural conditions. The phenotype may vary somewhat with variations in environment such as temperature, light intensity, length of day, irrigation amount and frequency, and/or fertilizer rate without, however, any variance in genotype.

The following traits have been repeatedly observed and are determined to be among the unique characteristics of ‘BERSUN’. These characteristics distinguish ‘BERSUN’ as a new and distinct cultivar:

1. Relatively compact growth habit; and

2. Distinctive leaf color pattern with its medium green foliage highlighted by a soft chartreuse variegation that may whiten when exposed to heat.

Plants of the new Hosta can be compared to plants of the Hosta fortunei cultivar ‘Francee’ (unpatented). Compared to plants of the cultivar ‘Francee’, the new Hosta is relatively more compact in its growth habit including shorter scapes with pale lavender flowers and foliage having a distinct color pattern including a soft chartreuse variegation and distinct edge coloring.

### BRIEF DESCRIPTION OF THE DRAWINGS

The invention is depicted in the accompanying colored photographs in which:

FIG. 1 shows the plant as a clump growing in a garden environment; and

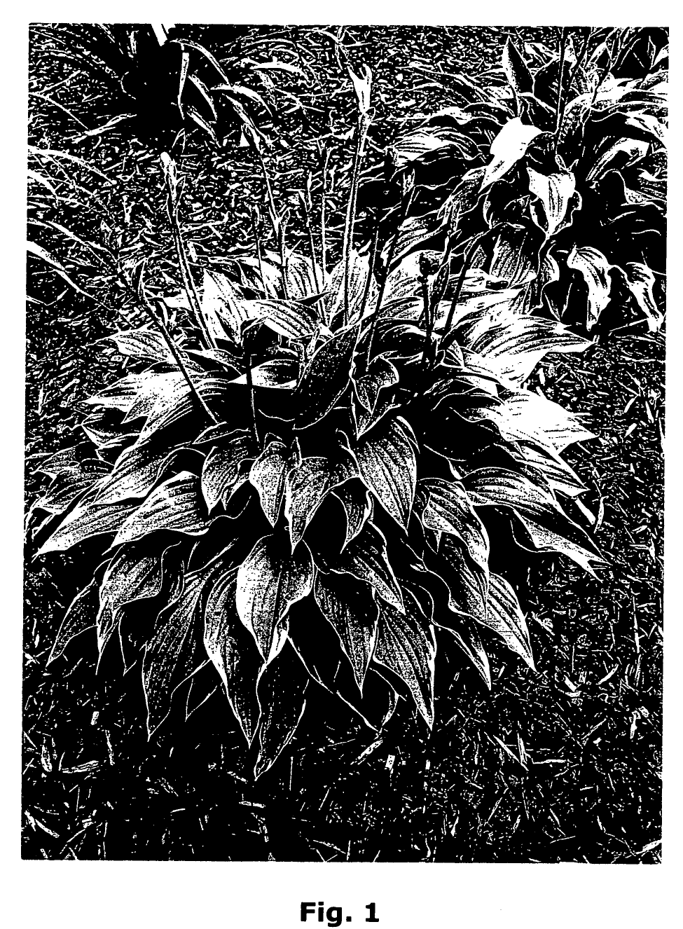

FIG. 2 is a side-by-side comparison of the new Hosta on the left with the comparator ‘Francee’.

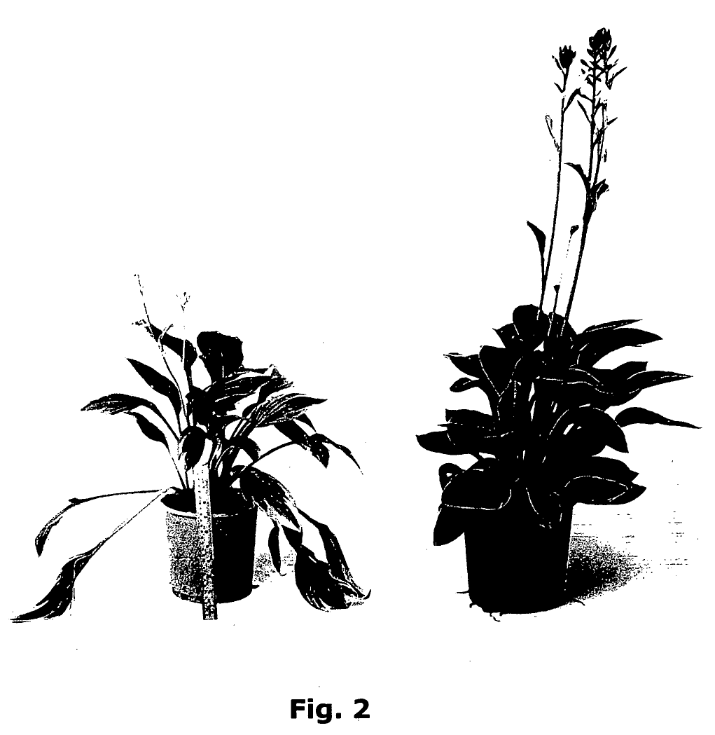

The photographs depict the overall appearance of the new Hosta, showing the colors as true as it is reasonably possible to obtain in colored reproductions of this type. Colors in the photographs may differ slightly from the color values cited in the Detailed Botanical Description which more accurately describe the actual colors of the new Hosta.

### DETAILED BOTANICAL DESCRIPTION OF THE NEW PLANT

The following detailed description sets forth characteristics of the new cultivar. The data which defines each characteristic was collected from asexual reproductions grown in an open greenhouse with irrigation provided by drip feeding in a hydroponic system, and carried out by the breeder at Clyde, Victoria, Australia, approximately 38 degrees, 9 minutes South latitude at 16 m elevation. The plant histories were taken on five plants of ‘BERSUN’ potted into 150 mm pots and four plants of ‘Francee’ potted into 200 mm pots.

In the following table of comparative features, color references are made to The Royal Horticultural Society of London Colour Chart, 1995 Edition. The color values were determined on plant material grown in an open greenhouse. The comparator description was taken from the Time-Life Encyclopaedia of Gardening.

| | ‘BERSUN’ | ‘Francee’ |
|---|---|---|
| The Plant | | |
| Plant Growth Rate | Moderately vigorous | Moderately vigorous |
| Length of Flowering | Mid-December/mid- | |
| Season | January through mid to | |
| | late March | |
| Time of Flowering | Mid-summer (southern | Mid-summer (southern |
| Season | hemisphere) | hemisphere) |
| Density | Moderate | Moderate |
| Plant Shape | Upright, irregularly | Upright, irregularly |
| | rounded | rounded |
| Growth Type | Clumping | Clumping |
| Mean Height (mm) | 332 | 392 |
| Mean Width (mm) | 634 | 554 |
| Leaf | | |
| Color-Main | 137B | Darker than 137B |
| Color-Edge | 150D | 2D |
| Characteristics | Petiolate | Petiolate |
| Mean Length (mm) | 188.4 | 195.4 |
| Mean Width (mm) | 78.24 | 93.08 |
| Apex | Pointed | Pointed |
| Margin | Undulation slight to | Undulation slight to very |
| | medium | slight |
| Texture | Prominently veined | Prominently veined |
| Shape | Petiolate to broad | Petiolate to broad |
| | lanceolate | lanceolate |
| Petiole | | |
| Size: Mean Length | 228 | 227 |
| (mm) | | |
| Size: Mean Width | 8.6 at mid-point | 11.98 at mid-point |
| | (mm) | |
| Color | 143C | 137C, edge 2D |
| Scapes | | |
| Branching | Non branching | Non branching |
| arrangement | sometimes with a leaf | sometimes with one to |
| | part way up | two leaves |
| Surface texture | Smooth | Smooth |
| Mean Length (mm) | 470 | 689.6 |
| Mean stem diameter | 3.74 | 6.3 |
| (mm) (just below | | |
| inflorescence) | | |
| INFLORESCENCE | | |
| Length of flowering | Mid-December/mid- | |
| period | January through mid to | |
| | late March | |
| Shape | Lily like | Lily like |
| Mean Number of | 12.2 | 22.8 |
| florets | | |
| Mean floret length | 57.54 | 55.74 |
| (mm) | | |
| Mean flower head size | 44.2 | 46.9 |
| (mm) | | |
| Lastingness of bloom | 2 days | 2 days |
| Fragrance of bloom | very weak and spicy | very weak |
| Profile Upper | Flat | Flat |
| Profile Lower | Wedge Shaped | Wedge Shaped |
| Color Midzone outside | 85D | 85C |
| Color Midzone inside | 85D | 85C |
| Color Margin inside | 85D | 85C |
| Flower bud | | |
| Shape | Elongated thin to an | Elongated thin to an |
| | ovate final ⅓^{rd} | ovate final ⅓^{rd} |
| Mean Length (mm) (at | 60.14 | 66.94 |
| time of sepal | | |
| separation) | | |
| Mean width (mm) (at | 13 | 11.6 |
| time of sepal | | |
| separation) | | |
| Color | 85A | 85A |
| Petal | | |
| Number | 6 | 6 |
| Mean length (mm) (as | 21.7 | 20.92 |
| at separation) | | |
| Mean width (mm) | 10.4 | 9.24 |
| Arrangement | 3 inner, 3 outer | 3 inner, 3 outer arranged |
| | arranged in opposition. | in opposition. Connected |
| | Connected at | at approximately |
| | approximately ⅓^{rd} | ⅓^{rd from apex} |
| from apex | | |
| Pedicel | | |
| Mean width (mm) | 2.38 | 2.38 |
| color | 85A | 85A |
| prickles | Nil | Nil |
| Mean Length (mm) | 7.18 | 11.6 |
| Basal Spot | | |
| Presence | Absent | Absent |
| Stamen | | |
| Filament color | 17B | 17C |
| Mean filament length | 4.98 | 2.88 |
| (mm) | | |
| Anther color | 145D | 145C at base to 145D at |
| | | top |
| Mean anther length | 56.18 (top bends back | 47 (top bent at right |
| (mm) (base to bend) | towards base) | angle) |
| Pistils | | |
| Mean style length | 67.1 (bent at right | 57.3 (bent at right angle |
| (mm) | angle at top) | at top) |
| Style color | 145D | 145C |
| Seed vessel | | |
| size | Small | Small |
| Color | 145B | 144A |
| Fruit Presence | 6 valved, loculicidal | 6 valved, loculicidal |
| | capsule | capsule |

### GENERAL CHARACTERISTICS

‘BERSUN’ is primarily distinguished from plants of the Hosta cultivar ‘Francee’ and other hybrid cultivars of Hosta genus by its uniquely compact growth habit and distinctive leaf color pattern. ‘BERSUN’ is known as a vigorous perennial with a compact growth habit and appealing medium green foliage highlighted by soft chartreuse variegation that may brighten to white when exposed to heat. An abundance of pale lavender flowers having a light, spicy fragrance frequently appear about mid-season on scapes rising about 18.5 inches (470 mm).

1. A new and distinct variety of Hosta plant, substantially as shown and described.
